(19) United States
(12) Patent Application Publication
(10) Pub. No.: US 2008/0112988 A1
(43) Pub. Date: May 15, 2008

(54) COMPOSITION AND METHOD FOR COSMETIC VITALIZATION AND STABILIZATION

(75) Inventors: Rondus Frank, Boulder Creek, CA (US); Cathlene Michaels-Brader, San Jose, CA (US); Gloria Webb-Williams, Fremont, CA (US)

Correspondence Address: ADVANTIA LAW GROUP, 9035 SOUTH 1300 EAST, SUITE 200, SANDY, UT, 84094, US

(73) Assignee: Ancient Stones, Inc., Santa Clara, CA (US)

(21) Appl. No.: 11/559,843

(22) Filed: Nov. 14, 2006

(51) Int. Cl. A61K 8/02; A61K 33/38; A61K 33/34; A61K 33/24; A61Q 19/00

(52) U.S. Cl. 424/401; 424/618; 424/630; 424/649

(57) ABSTRACT

A composition and method for vitalizing and stabilizing a cosmetic product, comprising: an inner cosmetic layer; and an outer shell including: metal ions; and an anhydrous cosmetic product. The metal ions are selected from the group consisting of: copper ions, silver ions, and gold ions. The concentration of the metal ions ranges from 2 ppm to 200 ppm. The two types of metal ions may be equal in concentration. Additionally, a composition for vitalizing and stabilizing cosmetics, includes: hydrophilic ions selected from the group consisting of copper ions, silver ions, and gold ions; and an anhydrous cosmetic product. The method includes: introducing hydrophilic ions into water substantially free of ions to form a solution; atomizing the solution; and combining the atomized solution with a cosmetic product. The method further includes inserting a pure sample of a desired element into the water substantially free of ions and running a current therethrough.

### BACKGROUND OF THE INVENTION

1. Field of the Invention

The present invention relates to topical compositions for cosmetic use, specifically to the combination of nano-particles with cosmetic products.

2. Description of the Related Art

In the related art, various topical creams, lotions, and other compositions are often used to vitalize, and stabilize a person's makeup and/or cosmetics after the makeup and/or cosmetics have been applied. It is also well understood in the art that makeup and/or cosmetics, once applied to a person's face and/or body, will frequently and most undesirably, rub off or mark other areas such as: clothing, furniture, other persons, and so forth. Furthermore, after application, and during the normal course of everyday use, the appearance of cosmetics and/or makeup may become insipid, uninspiring, and lackluster.

The cosmetic and/or makeup industry has applied ionic nano-particles in the form of lotions, powders and/or topical creams in attempts to resolve the aforementioned issues. Indeed, ionic nano-particles have been used in a variety of other fields, including: use for medical devices, water purification, sterilization, and so forth. However, many of the aforementioned uses combine the ionic nano-particles with other chemicals and/or additives; such as potentially harmful acids and other irritating surfactants. These chemicals and/or additives may cause irritation to the skin and/or cause allergic reactions.

Additionally, preparing and mixing these powders, creams, and lotions may be complicated, cumbersome, and require expensive chemicals, materials and/or components. Some improvements have been made in the field. Examples include but are not limited to the references described below, which references are incorporated by reference herein:

U.S. Pat. No. 6,720,006, issued to Hanke et al., discloses an anti-microbial body care product comprising in the part contacting the human or animal skin and/or mucosa and organic matrix containing homogeneously dispersed particles of a metallic silver having a particle size in the range of 1 to 50 nm (silver nano-particles) in an amount providing on the surface of said part and anti-microbially effective but less than cytotoxic silver concentration. Preferred body care products are disposable absorbent articles, toothbrushes, cosmetic products and baby comforting products.

U.S. Pat. No. 6,264,741, issued to Brinke et al., discloses a method of making a nano-composite self-assembly is provided where at least one hydrophilic compound, at least one hydrophobic compound, and at least one amphiphilic surfactant are mixed in an aqueous solvent with the solvent subsequently evaporated to form a self-assembled liquid crystalline mesophase material. Upon polymerization of the hydrophilic and hydrophobic compounds, a robust nanocomposite self-assembled material is formed. Importantly, in the reaction mixture, the amphiphilic surfactant has an initial concentration below the critical micelle concentration to allow formation of the liquid-phase micellar mesophase material. A variety of nano-composite structures can be formed, depending upon the solvent vaporization process, including layered mesophases.

U.S. Pat. No. 4,023,961, issued to Douglas et al., discloses a method of producing powdered material which includes the steps of providing at least one metal or its oxide in the form of a solution; forming the solution into an atomized spray; and reacting the atomized solution to produce a metal or metal oxide powder having a particle size which is determined by the composition of the solution in relation to the size of the individual droplets of the atomized spray.

U.S. Pat. No. 4,023,961, issued to Douglas et al., discloses a method of producing powdered material which includes the steps of providing at least one metal or its oxide in the form of a solution; forming the solution into an atomized spray; and reacting the atomized solution to produce a metal or metal oxide powder having a particle size which is determined by the composition of the solution in relation to the size of the individual droplets of the atomized spray.

U.S. Pat. No. 6,132,750, issued to Perrier et al., discloses an invention related to small sized particles. These particles comprise at least on the surface thereof a wall composed of a mixture of at least one protein and at least one polysaccharide which are cross-linked, preferably by interfacial cross-linking with a polyfunctional acylating agent which forms at least amide and ester bonds, and optionally anhydride bonds with amine, hydroxyl or carboxyl functions of the protein and of the polysaccharide, and which comprise hydroxamic groups on the surface thereof for chelating metal ions. These particles can be used in cosmetics or in pharmacy notably for the chelation or release of metal ions.

U.S. Pat. No. 6,514,406, issued to Katehis, discloses a water treatment apparatus comprises a first housing having an inlet, an outlet, and a chamber defined therebetween wherein the inlet is connected to an upstream water pipe. A series of magnets are arranged for attachment about the upstream water pipe for directing a magnetic field onto water flowing downstream into the first housing chamber. First and second electrodes of a copper and silver alloy are positioned in the first housing chamber for releasing copper and silver ions into the water therein to reduce bacteria levels of the water. A heating coil is disposed in the first housing for boiling the water in the first housing chamber. The apparatus includes a second housing having an inlet, an outlet, and a chamber defined therebetween. A conduit connects the outlet of the first housing with the inlet of the second housing for supplying water vapor from the first housing chamber to the second housing chamber. A funnel and a fan are positioned in the second housing chamber for condensing the water vapor into distilled water. The distilled water is funneled into a helical filter for producing filtered distilled water. The filtered water is supplied to a third housing for storage, the storage reservoir being connected to an outlet fluid pipe for supplying the filtered water to a faucet.

U.S. Pat. No. 5,662,913, issued to Capelli, discloses an antimicrobial metal-based compositions, which are photo-stable, non-staining, and which are easily absorbed into lipophilic matrices, contain silver cations, complexed by acyclic polyether polymers through the formation of a “host-guest relationship” where the acyclic polyether is the “host” and the silver cation is the “guest,” wherein stabilization of this “host-guest relationship” is accomplished through the use of excess anions.

U.S. Pat. No. 6,565,893, issued to Jones et al., discloses a process for preparing a disinfectant for use in swimming pools and the like. The process provides a formulation containing copper sulfate pentahydrate that will remain suspended so long as the formulation is maintained at a temperature above 40.degree. F. The process includes the step of mixing the copper sulfate pentahydrate with water, sulfuric acid and ammonium sulfate at a temperature of 187.degree. F.+−0.10.degree. F., then rapidly cooling the mixture to a temperature of 57-65.degree. F. Colloidal silver or gold added to the formulation before the step of rapidly cooling will also remain suspended. Hydrogen peroxide may be added to enhance the performance of the disinfectant.

U.S. Patent Application Publication No.: 2005/0139488, by Ogihara et al., discloses an electrolytic stripping method comprising electrolytically stripping: a copper plating film exposed from a silver plating film that partially covers a copper plating film formed on the entire surface of a member; and leaking silver formed on an exposed portion of the copper plating film and having a smaller thickness than the silver plating film, using the copper plating film as an anode, wherein the electrolytic stripping is carried out using a cyan compound-free electrolytic copper-stripping liquid, and the electrolytic stripping liquid comprises a compound capable of forming, with silver, a complex ion that is more easily dissociated than a complex ion of silver and cyan; and wherein silver and copper are deposited on a cathode which is used as a counter electrode to the anode and which is made of a metal that is chemically stable against the electrolytic stripping liquid.

U.S. Patent Application Publication No.: 2003/0036490, by Lorant et al., discloses an invention related to a composition for topical application, comprising at least one electrolyte and at least one amphiphilic polymer including at least one ethylenically unsaturated monomer containing a sulphonic group, in free form or partially or totally neutralized form and comprising at least one hydrophobic portion. The invention also relates to the uses of the said composition, especially in cosmetics, to treat and care for human skin, the scalp, mucous membranes, the nails and keratin fibers.

The inventions heretofore known suffer from a number of disadvantages which include: being expensive, being difficult and/or time consuming to produce, not applying well or mixing well with cosmetics and/or makeup, causing applied makeup to become powdery and/or runny, and/or requiring a user to apply multiple times. Furthermore, the compositions and methods heretofore known contain additional potentially toxic additives, such as acids and/or other chemicals.

What is needed is a cosmetic vitalization and stabilization formula that solves one or more of the problems described herein, and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available cosmetic compositions and accessories. Accordingly, the present invention has been developed to provide a cosmetic vitalization and stabilization composition and method that is simple and inexpensive to produce; does not contain potentially harmfully and/or corrosive chemicals; and/or mixes well with a cosmetic product. Further, the cosmetic vitalization and stabilization composition and method form a protective and lustrous shell over an applied cosmetic product. Additionally, the composition and method provide the ability for cosmetics to stay on the body and face without being powdery. The composition and method allow for one-time application of the composition for all day wear. The composition and method further enable a user to simply spray the composition on at the end of the day to revitalize and re-stabilize the cosmetics. This prevents the need for a user to reapply makeup and/or cosmetics at the end of the day.

In one embodiment, there is a composition for vitalizing and/or stabilizing an anhydrous cosmetic product. The composition may include metals ions and/or an anhydrous cosmetic product. The metal ions may be selected from the group consisting of copper ions, silver ions, and/or gold ions. In another embodiment, the composition may include copper ions and/or silver ions. In yet another embodiment, the metal ions may be equal in concentration. The concentration of metal ions may range from two parts per million (2 ppm) to two hundred parts per million (200 ppm).

In another embodiment, the composition for vitalizing and/or stabilizing an anhydrous cosmetic product may include hydrophilic ions selected from the group consisting of copper ions, silver ions, and/or gold ions, and/or an anhydrous cosmetic product. The method may further comprise introducing hydrophilic ions into water substantially free of ions to form a solution, wherein the hydrophilic ions may be selected from the group consisting of silver ions, copper ions, and/or gold ions. The method may further include atomizing the solution and/or then combining the atomized solution with a cosmetic product. The cosmetic product may include an anhydrous cosmetic product. The makeup product may also include an anhydrous mineral powder.

In still another embodiment, the concentration of hydrophilic ions may range from two parts per million (2 ppm) to two hundred parts per million (200 ppm). The solution may further include two or more types of hydrophilic ions. In an alternative embodiment, the hydrophilic ions may include gold ions and silver ions. The method may further include a solution wherein the hydrophilic ions may be copper ions and/or silver ions.

In yet another embodiment, the method may include introducing desired hydrophilic ions into water substantially free of ions. The method may include inserting a pure sample of a desired element into water substantially free of ions and running a current therethrough. The pure sample of a desired element may include a rod like shape. The method may further include applying a heat source to the solution while introducing hydrophilic ions into the water substantially free of ions. Additionally, the ionic solution may be placed in a spray bottle with a fine mist spray top. The solution may also be sprayed onto an applied cosmetic product, thereby atomizing the ionic solution.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

As used herein, “comprising,” “including,” “containing,” “is, are,” “characterized by,” and grammatical equivalents thereof are inclusive or open-ended terms that do not exclude additional un-recited elements or method steps. “Comprising” is to be interpreted as including the more restrictive terms “consisting of” and “consisting essentially of.”

In one embodiment, there is a composition for vitalizing and stabilizing a cosmetic product comprising an inner cosmetic layer and an outer shell. The outer shell includes metal ions and an anhydrous cosmetic product. The inner cosmetic layer may include any type of cosmetic product; such as, but not limited to, blush, powders, foundations, concealers, moisturizers, cover-ups, and/or primers. The inner cosmetic layer may include a variety of types of minerals. Some non-limiting examples of minerals include: mica, iron oxide, chromium oxide, standard titanium dioxide, nano-titanium dioxide, nano-sized zinc, Manganese Violet, and/or ultra marine blue.

In another embodiment, the composition includes an outer shell which is formed by the application of metal ions to the inner cosmetic layer. The outer shell includes metal ions and an anhydrous cosmetic product. The metals ions may be any type of hydrophilic ions. In an alternative embodiment, the metal ions may be selected from a group consisting of: gold ions, copper ions, and/or silver ions. The anhydrous cosmetic product may be any anhydrous cosmetic product contemplated in the art. Some non-limiting examples of an anhydrous cosmetic product include: powders, blushes, foundations, concealers, moisturizers, cover-ups, and/or primers. Additionally, cosmetic products which are anhydrous and contain mica, iron oxide, chromium oxide, standard titanium dioxide, nano-titanium dioxide, nano-sized zinc, Manganese Violet, and/or ultra marine blue may be used. Anhydrous is defined as being without water.

In yet another embodiment, the concentration of the metal ions ranges from two parts per million (2 ppm) to two hundred parts per million (200 ppm). Some non-limiting examples of ion concentrations include: at least about two parts per million (2 ppm) or about five parts per million (5 ppm), or about ten parts per million (10 ppm), or about twenty parts per million (20 ppm) or about forty parts per million (40 ppm), or about seventy parts per million (70 ppm); and less than about two hundred parts per million (200 ppm), or about one hundred seventy parts per million (170). One skilled in the art would recognize there is a multitude of techniques and/or instruments that will enable a user to make and/or determine a desired concentration of ions in solution. In one non-limiting example, a user may use a laboratory regulated parts per million meter to determine an ion concentration. Parts per million meters such as those available through Hanna Instruments from www.omnicontrols.com/Manufacturer_HANNA.aspx. Alternatively, a series of dilutions may further assist one skilled in the art to reach a desired concentration.

In yet another embodiment, the composition for vitalizing and stabilizing a cosmetic product may further include two or more types of ions selected from the group consisting of: gold ions, copper ions, and/or silver ions. These two or more types of ions may also be equal in concentration, as determined by the concentration ranges specified in the previous paragraphs.

In still another embodiment, the composition for vitalizing and stabilizing a cosmetic product includes hydrophilic ions selected from the group consisting of: copper ions, silver ions, and gold ions; as well as an anhydrous cosmetic product. Indeed, a single type of hydrophilic ion may be used and/or a combination of two or more ions may be used. Further, any anhydrous cosmetic product may be used. Some non-limiting examples of anhydrous cosmetics include: powders, blushes, foundations, concealers, moisturizers, cover-ups, and/or primers. Additionally, cosmetic products which are anhydrous and contain mica, iron oxide, chromium oxide, standard titanium dioxide, nano-titanium dioxide, nano-sized zinc, Manganese Violet, and/or ultra marine blue.

In another embodiment of the invention, there is a method of vitalizing and stabilizing a cosmetic product. The method includes: introducing hydrophilic ions into water substantially free of ions, forming a solution; atomizing the solution; and combining the atomized solution with a cosmetic product. Any type of water that is substantially free of ions and other particulates may be used. In being substantially free of ions, the water may be completely de-ionized, meaning not contain any ions. Alternatively, water may contain a small and/or minimal amount of ions and/or particulates. Ions are defined as any electrically charged atom (positive or negatively charged) or group of atoms formed by the loss or gain of one or more electrons. There exists a variety of types of water that are substantially free of ions from a variety of sources that would be appreciated by those skilled in the art. Some non-limiting examples of this type of water include: distilled water, de-ionized water, oxygenated biological water, and/or so forth.

In another embodiment, the hydrophilic ions introduced into the water may be selected from the group consisting of: silver ions, copper ions, and gold ions. Indeed, any combination of silver ions, copper ions, and gold ions may be introduced into the water substantially free of ions. For example, in one embodiment, the method may include introducing only silver ions into the water substantially free of ions. In another embodiment, the method may include introducing both silver ions and copper ions into the water substantially free of ions. In yet another embodiment, the method may include introducing both silver ions and gold ions into the water substantially free of ions. Further, the method may include introducing only one type ion from a group consisting of silver ions, copper ions, and/or gold ions into the water substantially free of ions.

In yet another embodiment, the method of vitalizing and stabilizing a cosmetic product may include a process for introducing the hydrophilic ions into the water substantially free of ions, thereby creating a solution of hydrophilic ions. A person skilled in the art would recognize there is a multitude of ways in which the hydrophilic ions may be introduced into solution. In one non-limiting example, a pure form of the desired metal ion, such as rods, is inserted into the water substantially free of ions. A low voltage direct current is then run through the rods, introducing the ions into solution.

In still another embodiment, the total concentration of the hydrophilic ions in the solution may range from two parts per million (2 ppm) to two hundred parts per million (200 ppm). Indeed, the total concentration of hydrophilic ions in the solution may range in concentration as described previously. A person skilled in the art would recognize there are many methods of ensuring and/or measuring the concentration levels in the solution. A non-limiting example includes using a laboratory regulated parts per million meter like those available through Hanna Instruments at www.omnicontrols.com/Manufacturer_HANNA.aspx. In an alternative embodiment, the concentration of two or more types of hydrophilic ions in the solution is equal, having a concentration ratio of 1:1. For example, the solution may contain an equal concentration of gold ions and silver ions.

In still another embodiment, the method of vitalizing and stabilizing a cosmetic product comprises mixing the solution with an anhydrous cosmetic product. The anhydrous cosmetic product may include any cosmetic product such as those described in the previous paragraphs.

In another embodiment, the method of vitalizing and stabilizing a cosmetic product includes applying a heat source while introducing the hydrophilic ions into solution. Applying the heat source may be accomplished any manner contemplated in the art or as described in the following paragraphs. However, the temperature of the place in which the solution is prepared should not exceed eighty degrees (80°) Fahrenheit.

In yet another embodiment, the method of vitalizing and stabilizing a cosmetic product includes placing the solution in a spray bottle; the spray bottle including a fine mist spray top. The solution may then be applied to a cosmetic product as by spraying through the fine mist top. This will atomize the solution, as the solution is being applied to a cosmetic product. Indeed, there is a multitude of ways in which the solution may be applied to a cosmetic product. However, in this embodiment, the spray top should be a fine mist spray top, such as those available from www.specialtybottle.com/? gclid=CNantoT7sog CFSdJYwodwUjjhw.

Advantageously, in one embodiment, the typical preparation of the composition for vitalizing and stabilizing a cosmetic product is both simple and easy. A solution of desired hydrophilic metal ions is prepared by taking a positive and a negative electrode and attaching each of them to the end of a pure metal sample of a desired hydrophilic metal ion, such as, but not limited to gold, silver, or copper. The other ends of the positive and negative electrodes are then connected to a low voltage continuous current machine. The connected metal samples are then inserted into a jar of distilled water, deionized water, and/or oxygenated biological water. Previously, the water should be tested for a baseline count of contents. The water should show either zero (0) or one (1) parts per million (ppm).

In another embodiment, a heat source is then applied to the jar of water. For example, the heat source may include a flame, such as a typical Bunsen burner and/or a simple light bulb of varying wattages, such 25 watts, 40 watts, 60 watts, etc. The low voltage continuous current machine is then turned on. The desired range for the voltage setting may be from thirty-one to thirty-five (31-35) volts. Indeed, the voltage may be at least about thirty-one (31) volts, but less than thirty-five (35) volts.

In one embodiment, direct current is then run through the metal samples from the low voltage continuous current machine until such time as the concentration of ions in the solution comes within the range of two parts per million (2 ppm) to two hundred parts per million (200 ppm). Some non-limiting examples of ion concentrations include: at least about two parts per million (2 ppm) or about five parts per million (5 ppm), or about ten parts per million (10 ppm), or about twenty parts per million (20 ppm) or about forty parts per million (40 ppm), or about seventy parts per million (70 ppm); and less than about two hundred parts per million (200 ppm) or about one hundred seventy parts per million (170). The concentration of ions in solution may be verified by using a simple laboratory regulated parts per million meter. Once a desired ion concentration is reached, the current running through the system should be discontinued and the solution should set for a period of time ranging from 10 seconds to 300 seconds. The ion concentrated water should then be filtered into a sterile jar. Any type of filter contemplated in the art may be used. In a non-limiting example, bleached and/or unbleached coffee filters may be used. Once filtered, the ionic solution should be re-measured to determine ion concentration. The result ionic solution should contain a concentration range of about 2 ppm to 200 ppm (or one of the ranges described previously) of one of the desired metal ions, such as gold ions, copper ions, or silver ions.

In yet another embodiment, two or more types of ions may be introduced into water substantially free of ions. The hydrophilic ions of each desired element are introduced into their own separate solutions in a similar manner as described above. For example, if a user desires to produce a hydrophilic solution with gold ions and silver ions, the user would create a separate solution of gold ions as described in the previous paragraph. The user would next create a separate solution of silver ions in a similar manner. Each separate solution of a desired hydrophilic ion may then be filtered and tested for a desired concentration. The separate hydrophilic solutions may then be combined and/or diluted to reach a desired concentration. A person skilled in the art would realize there are a variety of ways of obtaining a desired concentration, such as, but not limited to, creating various dilutions and testing for concentration.

In another embodiment, once a final hydrophilic solution is created with the desired concentration and/or types of hydrophilic ions, the solution is then atomized into a fine mist. Atomizing the solution may be done by placing the solution into a spray bottle that contains a fine mist spray top. The user sprays the solution onto a cosmetic product that has already been applied on the face and/or body. In the process of spraying the solution, the fine mist spray top atomizes the solution.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

For example, although the specification indicates the composition may be atomized and sprayed onto an applied cosmetic product, the composition may be mixed with a cosmetic product prior to a user applying the cosmetic product.

Additionally, although the specification indicates the use of jars in containing the ionic solution, it is contemplated a variety of container and/or jars may be used to mix and/or produce the solution.

Furthermore, the specification indicates the composition and/or method including: gold ions, silver ions, and/or copper ions. Gold ions may be defined as Gold (I) ions and/or Gold (III) ions. Silver ions may be defined as Silver (I) ions. Copper ions may be defined as Copper (I) and Copper (II) ions. Indeed, any potential ion of Gold, Silver, and/or Copper may be used.

It is also envisioned that the jars and/or containers may be a variety sizes and/or shapes. Some non-limiting examples include: wide-mouth jars, larger industrial containers, small or thin jars and/or containers, and/or so forth.

It is further envisioned the composition and method may include the use of colloidal minerals. The colloidal minerals may be combined with cosmetic product prior to a user's application of the cosmetic product. The colloidal minerals may further be combined with a metal ion to enhance the stabilization process.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

What is claimed is:

1. A composition for vitalizing and stabilizing, comprising:
an inner cosmetic layer;
an outer shell including:
metal ions; and
an anhydrous cosmetic product.

2. The composition of claim 1, wherein the metal ions are selected from the group consisting of: copper ions, silver ions, and gold ions.

3. The composition of claim 2, wherein the concentration of the metal ions ranges from 2 ppm to 200 ppm.

4. The composition of claim 3, further comprising two types of metal ions selected from the group consisting of: copper ions, silver ions, and gold ions.

5. The composition of claim 4, wherein the two types of metal ions are equal in concentration.

6. The composition of claim 2, wherein the metal ions include copper ions and silver ions.

7. A composition for vitalizing and stabilizing cosmetics, comprising:
hydrophilic ions selected from the group consisting of copper ions, silver ions, and gold ions; and
an anhydrous cosmetic product.

8. The composition of claim 7, wherein the concentration of the metal ions ranges from 2 ppm to 200 ppm.

9. The composition of claim 7, further comprising two types of metal ions selected from the group consisting of: copper ions, silver ions, and gold ions.

10. The composition of claim 9, wherein the two types of metal ions are equal in concentration.

11. The formula of claim 9, wherein the two types of ions are gold ions and silver ions.

12. A method for vitalizing and stabilizing a cosmetic product comprising:
introducing hydrophilic ions into water substantially free of ions to form a solution, wherein the hydrophilic ions are selected from the group consisting of silver ions, copper ions, and gold ions;
atomizing the solution; and
combining the atomized solution with a cosmetic product.

13. The method of claim 12, wherein the cosmetic product comprises an anhydrous cosmetic product.

14. The method of claim 12, wherein the concentration of hydrophilic ions ranges from two parts per million (2 ppm) to two hundred parts per million (200 ppm).

15. The method of claim 14, wherein the solution includes two or more types of hydrophilic ions.

16. The method of claim 15, wherein the solution includes gold ions and silver ions.

17. The method of claim 13, wherein the solution includes copper and silver ions.

18. The method of claim 10, wherein the step of introducing desired hydrophilic ions into water substantially free of ions further includes inserting a pure sample of a desired element into the water substantially free of ions and running a current therethrough.

19. The method of claim 12, further comprising applying a heat source while introducing hydrophilic ions into the water substantially free of ions.

20. The method of claim 12, furthering comprising placing the solution in a spray bottle; atomizing the solution by spraying the solution with a fine mist spray top; and combining the atomized solution with an anhydrous cosmetic product.
